(19) United States
(12) Patent Application Publication
(10) Pub. No.: US 2009/0180587 A1
(43) Pub. Date: Jul. 16, 2009

(54) METHOD OF DETECTING FINE SURFACE DEFECTS

(76) Inventors: Sang Hee NAM, Busan (KR); Sang Sik KANG, Gyeongsangnam-do (KR); Sung Ho CHO, Busan (KR); Choul KWON, Busan (KR); Min Seok YUN, Busan (KR); Kyung Min OH, Busan (KR)

Correspondence Address: BIRCH STEWART KOLASCH & BIRCH, PO BOX 747, FALLS CHURCH, VA, 22040-0747, US

(21) Appl. No.: 12/351,119

(22) Filed: Jan. 9, 2009

(30) Foreign Application Priority Data
Jan. 10, 2008 (KR) 10-2008-0003118

(51) Int. Cl. G01N 23/223; G01N 33/28

(52) U.S. Cl. 378/44; 356/38

(57) ABSTRACT

Disclosed herein is a method of detecting fine surface defects, including: applying nano phosphor paste on a surface of a subject; squeegeeing the surface of the subject such that the nano phosphor paste remains only in defects in the subject; and detecting the fine defects in the subject by placing the subject over a light detection plate, irradiating the subject with a light source, and then determining that light-emitting portions detected using the light detection plate are defects in the subject. The method of detecting fine surface defects is advantageous in that the problems with the conventional methods, such as surface contamination, the difficulty in realizing uniform application of a penetrant on a subject, the limitations on the dwelling time of the penetrant and the testing temperature (surface temperature of the subject: 16˜50° C.) thereof, and the contamination of the penetrant, can be overcome, and in that the incidence of inspection errors is decreased, and the time and cost of inspection are reduced, thus improving the production yield of the subject.

### BACKGROUND OF THE INVENTION

1. Field of the Invention

The present invention relates to a method of detecting fine surface defects, and, more particularly, to a method of detecting fine surface defects formed on the surface of spectacles, lenses, flat glass and TFT arrays used for radiation detectors using nano phosphor (that is, nano fluorescent material), by which defects that cannot be visually observed are detected, so that the yield of products increases and the inspection time thereof decreases, thereby improving productivity.

2. Description of the Related Art

Conventionally, a liquid penetrant examination method has been used as a method of detecting fine surface defects. This liquid penetrant examination method, which is in the field of nondestructive inspection, is a method of evaluating the quality of products by examining the position, size and patterns of the defects formed on the products, including the processes of applying a penetrant on the surface of a test piece such that the penetrant infiltrates into the defects, such as cracks, fissures, crevices, and the like, of the test piece, removing the excess penetrant remaining on the surface of the test piece, and applying a developer thereon to extract the infiltrated penetrant therefrom.

The penetrant test for finding the fine surface defects in the test piece is conducted using a capillary phenomenon.

Further, as methods of applying a penetrant on the surface defects in a subject, a method of spraying a penetrant on the surface defects in a subject using a sprayer and a method of spreading a penetrant on the surface defects in a subject using a predetermined brush have been used.

However, the method of spraying a penetrant on the surface defects in a subject using a sprayer is problematic in that the penetrant is also sprayed on unwanted areas, and it is not easy to consistently apply the penetrant on the subject. Further, the method is problem in that the dwelling time of the penetrant and the testing temperature (surface temperature of the subject: 16˜50° C.) thereof are limited, and the penetrant is subject to contamination.

Similarly, the method of spreading a penetrant on the surface defects in a subject using a predetermined brush is also problematic in that it is not easy to consistently apply the penetrant on the subject.

Therefore, conventional methods of detecting fine surface defects are problematic in that the inspection efficiency of fine surface defects is low.

### SUMMARY OF THE INVENTION

Accordingly, the present invention has been made to solve the above problems occurring in the prior art, and an object of the present invention is to provide a method of detecting fine surface defects which can overcome the problems with the conventional methods, such as surface contamination, the difficulty in realizing uniform application of a penetrant on a subject, the limitations on the dwelling time of the penetrant and the testing temperature (surface temperature of the subject: 16˜50° C.) thereof, and the contamination of the penetrant, and which can improve the inspection efficiency of fine surface defects.

In order to accomplish the above object, the present invention provides a method of detecting fine surface defects, including: applying nano phosphor paste on the surface of a subject; squeegeeing the surface of the subject such that the nano phosphor paste remains only in defects in the subject; and detecting defects in the subject by placing the subject over a light detection plate, irradiating the subject with a light source, and then determining that light-emitting portions, detected using the light detection plate, are defects in the subject.

In the method, the nano phosphor paste may be one or more selected from among Gd₂O₃:Eu; (Cd,Sr)TiO₃:Pr; Y₂O₃:Eu; Y₂O₃S:Eu; Zn(Ga,Al)₂O₄:Mn; Y₃ (Al,Ga)₅O₁₂:Tb; Y₂SiO₅:Tb; ZnS:Cu,Al; Y₂SiO₅:Ce; ZnGa₂O₄; ZnS:Ag,Cl; and combinations thereof.

Further, in the detecting the defects in the subject, information on the position, shape and degree of the defects in the subject may be obtained by detecting the light emitted from the nano phosphor paste, which reacts to X-rays, remaining in the defects using X-rays as the light source.

### BRIEF DESCRIPTION OF THE DRAWINGS

The above and other objects, features and advantages of the present invention will be more clearly understood from the following detailed description taken in conjunction with the accompanying drawing, in which:

FIG. 1 is a process view showing a method of detecting fine surface defects according to the present invention.

### DESCRIPTION OF THE PREFERRED EMBODIMENTS

Hereinafter, the present invention will be described in detail with reference to the attached drawing. In the description of the present invention, when it is determined that the technical point of the present invention would be made unclear by a detailed description of commonly-known technologies, such detailed description of commonly-known technologies will be omitted.

Further, the terms used herein are defined in consideration of the functions in the present invention, and can be changed depending on the intentions of users, or precedents. Therefore, the definition of the following terms must be understood based on the entire content of the specification of the present invention.

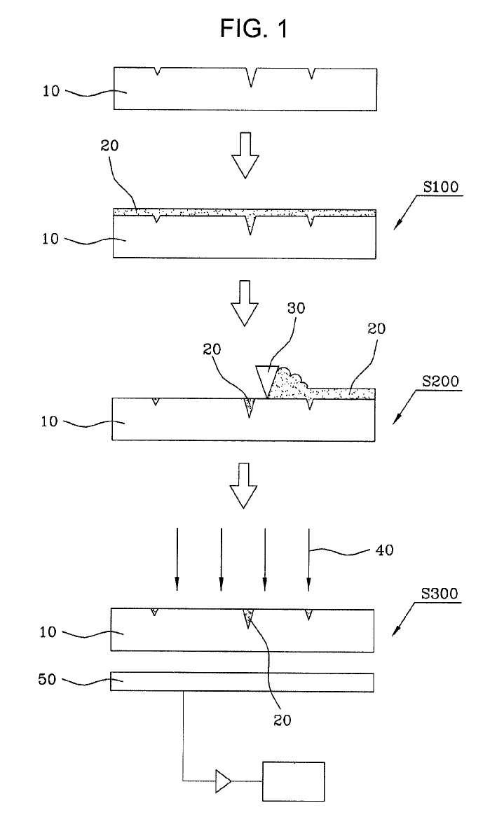

FIG. 1 is a process view showing a method of detecting fine surface defects according to the present invention.

As shown in FIG. 1, the method of detecting fine surface defects according to the present invention includes the steps of applying (S100), squeegeeing (S200) and detecting (S300).

In the step of applying (S100), nano phosphor paste (i.e., nano fluorescent paste) 20 is applied on a subject 10.

Generally, phosphor is used in a nanopaste state. In the present invention, the phosphor in a nanopaste state is applied on the surface of the subject 10.

In this case, the subject 10 may be LCD glass substrates, lenses, and substrates similar to these, and particularly, flat substrates, which require high surface flatness and are seriously deteriorated by surface defects, and substrates similar to these.

Since the phosphor, i.e., the fluorescent material, used in the step of applying (S100) is in a nanopaste state, when the phosphor is applied on the surface of the flat substrate, it infiltrates into fine defects formed on the surface of the flat substrate, as shown in FIG. 1.

In the step of squeegeeing (S200), the surface of the subject 10 is squeegeed such that the nano phosphor paste remains only in fine defects in the subject.

The squeegeeing the surface of the subject 10 means that nano phosphor paste 20 is scraped away from the surface of the subject 10 by scraping the surface of the subject 10 using a squeegee 30. That is, when the surface of the subject 10 is squeegeed, the nano phosphor paste 20 remaining on the surface of the subject 10 is removed, and only the nano phosphor paste 20 that has infiltrated into the fine defects in the subject 10 remains.

Generally, the squeegeeing work is conducted to form a thin film on a substrate using nano paste, but, in the present invention, it is conducted to remove nano paste from the surface of a subject.

In the step of detecting (S300), the subject 10 is placed over a light detection plate 50, the subject 10 is irradiated with a light source, and then it is determined that light-emitting portions detected using the light detection plate 50 are fine defects in the subject.

Generally, phosphor has reaction characteristics in a wide wavelength region ranging from radiation such as X-rays, X-rays, and the like to visible rays, and, particularly, has the best reaction characteristics in the X-ray region.

Therefore, when X-rays 40 are used as the light source in the step of detecting (S300), the phosphor remaining in the fine defects in the subject exhibits excellent luminescence reaction characteristics by X-rays, also exhibits excellent luminescence reaction characteristics even when a small amount of X-rays is applied, can be used in a dark room, and is well matched with the light detection plate 50.

The phosphor exhibiting such reaction characteristics may include Gd₂O₃:Eu; (Cd,Sr)TiO₃:Pr; Y₂O₃:Eu; Y₂O₃S:Eu; Zn(Ga,Al)₂O₄:Mn; Y₃ (Al,Ga)₅O₁₂:Tb; Y₂SiO₅:Tb; ZnS:Cu,Al; Y₂SiO₅:Ce; ZnGa₂O₄; ZnS:Ag,Cl; and the like. In the present invention, the nano phosphor paste 20 may be one or more selected from among Gd₂O₃:Eu; (Cd,Sr)TiO₃:Pr; Y₂O₃:Eu; Y₂O₃S:Eu; Zn(Ga,Al)₂O₄:Mn; Y₃(Al,Ga)₅O₁₂:Tb; Y₂SiO₅:Tb; ZnS:Cu,Al; Y₂SiO₅:Ce; ZnGa₂O₄; ZnS:Ag,Cl; and combinations thereof.

That is, the nano phosphor paste 20 that has infiltrated in the fine defects in the subject 10 in the step of detecting (S300) emits light by a light source. It is preferred that X-rays, which can generate higher energy than the band gap energy of phosphor and thus can excite the phosphor, be used as the light source.

The light emitted from the excited phosphor is detected using the light detection plate 50 placed under the subject 10, and is then converted into electrical signals to find information on light intensity and light position through the electrical signals. Base on the information, the degree and position of the defects in the subject are observed, and thus whether or not the subject is defective is determined.

As described above, the method of detecting fine surface defects according to the present invention is advantageous in that the problems with the conventional methods, such as surface contamination, the difficulty in realizing uniform application of a penetrant to a subject, the limitations on the dwelling time of the penetrant and the testing temperature (surface temperature of the subject: 16˜50° C.) thereof, and the contamination of the penetrant, can be overcome.

Further, the method of detecting fine surface defects according to the present invention is advantageous in that the incidence of inspection errors is decreased, and the time and cost of inspection are reduced, thus improving productivity.

As described above, since the preferred embodiment of present invention is only an example for realizing the present invention, various combinations are possible to realize the technical point of the present invention.

Therefore, although the preferred embodiment of the present invention has been disclosed for illustrative purposes, those skilled in the art will appreciate that various modifications, additions and substitutions are possible, without departing from the scope and spirit of the invention as disclosed in the accompanying claims.

What is claimed is:

1. A method of detecting fine surface defects, comprising:
applying nano phosphor paste on a surface of a subject;
squeegeeing the surface of the subject such that the nano phosphor paste remains only in defects in the subject; and
detecting the defects in the subject by placing the subject over a light detection plate, irradiating the subject with a light source, and then determining that light-emitting portions detected using the light detection plate are defects in the subject.

2. The method of detecting fine surface defects according to claim 1,
wherein the nano phosphor paste is one or more selected from among Gd₂O₃:Eu; (Cd,Sr)TiO₃:Pr; Y₂O₃:Eu; Y₂O₃S:Eu; Zn(Ga,Al)₂O₄:Mn; Y₃ (Al,Ga)₅O₁₂:Tb; Y₂SiO₅:Tb; ZnS:Cu,Al; Y₂SiO₅:Ce; ZnGa₂O₄; ZnS:Ag,Cl; and combinations thereof.

3. The method of detecting fine surface defects according to claim 1, wherein, in the detecting the defects in the subject, information on the position, shape and degree of the defects in the subject is obtained by detecting the light emitted from the nano phosphor paste, which reacts to X-rays, remaining in the defects using X-rays as the light source.

4. The method of detecting fine surface defects according to claim 2, wherein, in the detecting the defects in the subject, information on the position, shape and degree of the defects in the subject is obtained by detecting the light emitted from the nano phosphor paste, which reacts to X-rays, remaining in the defects using X-rays as the light source.
